(19) United States
(12) Patent Application Publication
(10) Pub. No.: US 2023/0295670 A1
(43) Pub. Date: Sep. 21, 2023

(54) METHODS FOR THE BIOCATALYTIC PRODUCTION OF ACETALDEHYDE

(71) Applicant: University of Kansas, Lawrence, KS (US)

(72) Inventors: Alan Martin Allgeier, Lawrence, KS (US); Victor Kumar Sharma, Lawrence, KS (US); Thomas Binder, Marion, IA (US)

(21) Appl. No.: 18/185,820

(22) Filed: Mar. 17, 2023

Related U.S. Application Data
(60) Provisional application No. 63/321,432, filed on Mar. 18, 2022.

(51) Int. Cl. C12P 7/26; C12N 9/04

(52) U.S. Cl. CPC C12P 7/26; C12N 9/0006; C12Y 101/01001

(57) ABSTRACT

Methods for producing acetaldehyde from ethanol are provided. In embodiments, such a method comprises (a) exposing ethanol and furfural to a biocatalyst comprising yeast alcohol dehydrogenase 1, yeast alcohol dehydrogenase 2, and yeast alcohol dehydrogenase 3, and a biocatalyst cofactor under conditions that oxidize the ethanol to acetaldehyde and reduce the furfural to furfuryl alcohol to provide a product mixture comprising the acetaldehyde and the furfuryl alcohol; and (b) recovering the acetaldehyde from the product mixture as it is being produced in step (a).

### CROSS-REFERENCE TO RELATED APPLICATIONS

The present application claims priority to U.S. Provisional Patent Application No. 63/321,432 that was filed Mar. 18, 2022, the entire disclosure of which is hereby incorporated by reference.

### BACKGROUND

Acetaldehyde is a chemical intermediate produced from petroleum or coal fossil resources at 1200 kton/year, plus a large captive production and use. Chemocatalytic methods of acetaldehyde production are known, but they not applicable to low temperature processing associated with biocatalysts. Oxidoreductases constitute a family of biocatalysts known for catalyzing an array of biologically relevant redox reactions. Owing to their efficient redox capabilities, they have been of high interest for applications in certain industries like pharmaceuticals, biofuels, cosmetics and nutraceuticals among others. A certain class of oxidoreductases, alcohol dehydrogenases (EC 1.1.1.1), catalyze the production of valuable alcohols, ketones, aldehydes and amines. Alcohol dehydrogenases (ADHs) are NAD(P) coenzyme dependent oxidoreductases with the ability to catalyze interconversion of alcohols to aldehyde and; or ketones. Recycling the expensive cofactors is pertinent for practical applications of the enzyme as these cofactors are required in stoichiometric quantities. Establishing a biocatalytic redox cascade by combining individual oxidation and reduction reactions is often deterred by thermodynamic limitation due to insufficient Gibbs free energy change of the resultant reaction. The selection of a viable coproduct and cascade process is a notable challenge, given the specificity of enzymes but is essential for the practical utilization of the expensive cofactors,

### SUMMARY

Provided herein are methods for producing acetaldehyde from ethanol. The methods involve the co-conversion of ethanol to acetaldehyde and furfural to furfuryl alcohol using yeast alcohol dehydrogenases. Surprisingly high reactant conversions and product selectivities are achieved. For example, although furfural conversion to furfuryl alcohol is generally considered inhibitory to yeast and yeast alcohol dehydrogenase isozyme 1 is considered a low-efficiency enzyme, embodiments of the present methods achieve approximately 70% equilibrium conversion of furfural with nearly 100% selectivity to furfuryl alcohol under room temperature and wide range of pH conditions. The corresponding selectivity to the co-product acetaldehyde is nearly 100%, when the ethanol is supplied at 4:1 ratio compared to the furfural.

Methods for producing acetaldehyde from ethanol are provided. In embodiments, such a method comprises (a) exposing ethanol and furfural to a biocatalyst comprising yeast alcohol dehydrogenase 1, yeast alcohol dehydrogenase 2, and yeast alcohol dehydrogenase 3, and a biocatalyst cofactor under conditions that oxidize the ethanol to acetaldehyde and reduce the furfural to furfuryl alcohol to provide a product mixture comprising the acetaldehyde and the furfuryl alcohol; and (b) recovering the acetaldehyde from the product mixture as it is being produced in step (a).

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIG. 1 is a schematic illustration of the mechanism of yeast alcohol dehydrogenase (YADH) catalyzed co-production of acetaldehyde from ethanol and furfuryl alcohol from furfural.

FIG. 2 shows a chromatogram of a product mixture from an embodiment of the present methods. Retention times are 1.07=acetaldehyde, 2.40=ethanol, 8.05=internal standard, 8,67=furfural, 9.97==furfuryl alcohol,

FIG. 3 shows the residual activity of an immobilized biocatalyst (YADH@FAL/GA/PA110) over consecutive activity assays.

FIG. 4A plots the conversion of ethanol and furfural obtained from consecutive batch reactions utilizing the immobilized biocatalyst of FIG. 3. FIG. 4B plots the same conversion of ethanol and furfural but as a function of the number of hours in operation.

### DETAILED DESCRIPTION

Provided herein are methods for producing acetaldehyde. In the methods, ethanol and furfural are exposed to a biocatalyst and a biocatalyst cofactor under conditions sufficient to both oxidize the ethanol to acetaldehyde and reduce the furfural to furfuryl alcohol. The redox reactions catalyzed by the biocatalyst cofactor take place in a reaction mixture comprising (or consisting of) the ethanol, the furfural, the biocatalyst, the cofactor, and generally a solvent, e.g., water (and ultimately, products of the redox reactions). Other components may also be present in the reaction mixture, e.g., a buffer and/or a pH adjusting agent. The biocatalyst comprises (or consists of) three yeast alcohol dehydrogenases, yeast alcohol dehydrogenase 1 (YADH1), yeast alcohol dehydrogenase 2 (YADH2), and yeast alcohol dehydrogenase 3 (YADH3). The term “YADH” may be used in reference to the biocatalyst comprising (or consisting of) the three isozymes. These yeast alcohol dehydrogenases may be obtained from Saccharomyces cerevisiae that has not been subjected to any genetic modifications. The cofactor may comprise (or consist of) nicotinamide adenine dinucleotide (NAD) or its phosphate derivative, nicotinamide adenine dinucleotide phosphate (NADP), The term “NAD” may be used in reference to either or both forms of NAD, NAD+ and NADH, The sources of the ethanol and the furfural being converted by the present methods are not particularly limited. Illustrative sources include a lignocellulosic biomass such as corn, wheat, sugarcane, etc.

The exposure of the ethanol and the furfural to the biocatalyst/cofactor may be carried out in various ways, e.g., by adding the biocatalyst to a reactant mixture comprising the ethanol, the furfural, and the solvent. The cofactor may be added with the biocatalyst or may have been previously added to the reactant mixture. As noted above, the composition comprising the co-reactants (ethanollfurfural) as well as the biocatalyst/biocatalyst cofactor is referred to as the reaction mixture.

The present methods may be carried out in a variety of reactor systems, including batch reactor systems, semi-continuous flow reactor systems, and continuous flow reactor systems. Packed bed reactor systems, fluidized bed reactor systems, and continuously stirred tank reactor systems may be used. The reaction mixture may be in the form of a flowing reaction mixture stream, e.g., when using semi-continuous or continuous flow reactor systems.

The conditions under which the exposure of the co-reactants to the biocatalyst/biocatalyst cofactor takes place include parameters such as the amounts of the ethanol, the furfural, the biocatalyst, and the biocatalyst cofactor in the reaction mixture; the pH of the reaction mixture; the temperature of the reaction mixture; and the length of exposure. These conditions may be adjusted to achieve a desired reactant conversion and/or product selectivity. Regarding amounts, the ethanol and the furfural may be present in the reaction mixture at a concentration ratio of from 0.5:1 to 10:1 and more preferably between 2:1 to 10:1 to achieve high furfural conversion. The biocatalyst may be present in the reaction mixture at a concentration of from 0.05 mg/ml to 0.3 mg/ml regardless of the isozymes' composition. The biocatalyst cofactor may be present in the reaction mixture at a concentration of from 0.1 trill to 1 mM. The reaction mixture may have a pH in a range of from 6.5 to 9, preferably between pH 7 and 8.5. The pH may be controlled via a buffer (as described above) via monitoring and control of the pH of the reaction mixture by addition of acidic or basic additives (e.g., NaOH), necessary to maintain the pH. Mild temperatures may be used, including room temperature (20° C. to 2° C.) and up to 40° C. The exposure may be carried out for a period of time from 2 h to 24 h (enzyme activity may decrease over time on exposure).

In the present methods, the biocatalyst may be provided as an immobilized biocatalyst comprising (or consisting of) a solid support and the biocatalyst immobilized thereon. The immobilization may be via non-covalent or covalent bonding to the solid support. That is, the biocatalyst may be attached to the solid support through covalent bonds, non-covalent bonds, or both. This attachment may involve the use of a linking molecule capable of attaching (via covalent and/or non-covalent bonds) to both the sol id support and the biocatalyst. Various materials may be used for the solid support, e.g., a metal oxide, a zeolite, a mesoporous silicate, or a polymer. Illustrative polymers include polystyrene, poly acrylate, cross-linked polyethylene glycol, an alignate, chitosan, or a polysaccharide. The solid support may be in the form of a plurality of particles such that it is porous. The solid support may be functionalized (e.g., functionalized polystyrene, functionalized polyacrylate) to provide functional groups capable of forming the covalent and/or non-covalent bonds directly to the biocatalyst or indirectly, via the linking molecules noted above. Such functional groups comprise aldehyde groups, alkyne groups, azide groups, carboxylate groups, anhydride groups, amine groups, and combinations thereof. Illustrative linking molecules include glutaraldehyde, succinaldehyde, adipaldehyde, terepthaldehyde, propyne, propylazide, terephthalate esters, adipic acid, terephthaloyl chloride, adipoyl chloride, maleic anhydride, phthalic anhydride, carbomethoxymaleic anhydride, acrylonitrile, propylamine, pentamethylene diamine, and hexamethylene diamine.

Immobilized biocatalysts may be synthesized by contacting the biocatalyst with the selected solid support in a fluid comprising water and under conditions to induce the attachment via the covalent and/or non-covalent bonds. This may include use of a catalyst or an acid or a base. The temperature may be from −5 to 60° C., including from 20 to 40° C. Immobilized biocatalysts may be used in any of the reactor systems described above.

Example 3, below, describes the immobilization of YADH on a glutaraldehyde-functionalized polystyrene solid support. Briefly, the solid support was formed by combining polystyrene (previously functionalized with amine groups) and glutaraldehyde under conditions to react amine groups on the polystyrene with aldehyde groups on the glutaraldehyde so as to covalently bind the glutaraldehyde to the polystyrene via. imine linkages. Next, remaining amine groups on the glutaraldehyde-functionalized polystyrene solid support were capped with furfural. Finally, this capped, glutaraldehyde-functionalized polystyrene solid support was then contacted with YADH wider conditions to induce the reaction of free amine groups on YADH (e.g., from lysine and arginine) with unreacted aldehyde groups on the covalently bound glutaraldehyde linking molecules. Example 3 further describes the use of the resulting immobilized biocatalyst in the redox co-conversion of ethanol to acetaldehyde and furfural to furfuryl alcohol over extended periods of time.

Exposure under the conditions described above results in the co-production of the co-products acetaldehyde and furfuryl alcohol. For clarity, at this point, the reaction mixture comprising at least some amount of the co-product(s) may be referred to as a product mixture, with the understanding that generally, some amount of the co-reactants are also present in this product mixture. As noted above with respect to the reaction mixture, the product mixture may be in the form of flowing product mixture stream. The co-products of the redox reactions induced by the biocatalyst/biocatalyst cofactor may be recovered from the product mixture and if desired, used for other purposes, e.g., the synthesis of other chemicals. Thus, the present methods may include one or both of these additional steps.

Regarding the recovery of a co-product, e.g., acetaldehyde, such recovery may be coupled to the redox conversions taking place. That is, the co-products) may be recovered from the product mixture as they are being produced, i.e., simultaneously. For example, acetaldehyde may be simultaneously recovered from the product mixture as it is being produced from the oxidation of ethanol. This shifts the equilibrium redox reaction to a higher conversion of furfural and thus, greater yields, consistent with Le Chatlier's principle. Co-product(s) recovery, including simultaneous recovery, may be carried out using the technique described below and further illustrated in Example 2.

In embodiments, simultaneous recovery of acetaldehyde from a product mixture is carried out by introducing a gas to a product mixture comprising the acetaldehyde under conditions to transfer at least a portion of the acetaldehyde from the product mixture to the gas to provide an effluent gas comprising the acetaldehyde, and collecting the effluent gas. In addition to the acetaldehyde, the product mixture generally comprises the other components described above, e.g., furfuryl alcohol, ethanol, furfural, biocatalyst, biocatalyst cofactor, solvent(s), and buffer(s) and/or pH adjusting agent(s). Like the reaction mixture, the product mixture is a liquid. The product mixture may comprise these components at various amounts depending upon initial amounts used in the reaction mixture and the exposure conditions used to induce the redox reactions as described above. In embodiments, the product mixture comprises up to I M acetaldehyde, up to 1 M furfuryl alcohol, up to 2.5 M ethanol, and up to 0.5 M furfural, This includes, e.g., from 0.03 to 0.3 M acetaldehyde, from 0.03 to 0.3 M furfuryl alcohol, from 0.25 to 1 M ethanol, and from 0.005 to 0.04 M furfural.

The gas used for simultaneous recovery of the co-product(s) may be provided as a flowing gas stream. The gas is desirably an oxygen free gas, i.e., the gas does not comprise oxygen. However, the amount of oxygen need not be perfectly zero as a minor amount of oxygen may be present without any material effect on the simultaneous recovery step. An “oxygen free gas” may comprise less than 2% O₂, less than 1% O₂, or less than 0.1% 02. Otherwise, the gas may comprise (or consist of) a single type of gas or a gas mixture comprising different types of gases. The gas(es) may be selected from inert gases and noble gases. Illustrative gases include N₂, Ar, CO₂, and combinations thereof. The conditions under which the gas is introduced to achieve simultaneous recovery of the co-product(s) include parameters such as the gas composition, the gas flow rate, and the gas pressure. Since coupling the recovery of the co-product(s) to the redox conversion of the co-reactants shifts the equilibrium reaction, these conditions may be adjusted to achieve a desired co-reactant conversion and/or co-product selectivity. Illustrative gas flow rates include those in a range of from 0.1 to 120.0 standard liters per minute per kg of the product mixture. This includes from 0.6 to 60.0 standard liters per minute per kg of the product mixture. Illustrative gas pressures include those in a range of from 0.1 to 4 attn. This includes from 0.7 to 1.5 atm. It will be noted that distillative removal of acetaldehyde within these pressure ranges is within scope of the disclosure, de facto, as it is a method to remove acetaldehyde to the vapor phase. The temperature may he within the range of those described above with respect to the reaction mixture. Use of simultaneous recovery of acetaldehyde from a product mixture as it is being produced from the oxidation of ethanol, with an accompanying increase in the productivity of acetaldehyde generation is further illustrated in Example 2, below.

The present methods may be characterized by a conversion of a particular reactant to products. By conversion of each reactive component, it is meant (initial concentration-final concentration)*100/initial concentration of the reactive component. For ethanol, the conversion may be at least 10% to up to 60%. For furfural, the conversion may be at least 20% to up to 100%. Both estimates are subject to ratio of initial ethanol and furfural concentrations.

The present methods may be characterized by a selectivity for a particular product. By selectivity of each product component, it is meant (moles of product produced/moles of the corresponding reactive component consumed)*100, For acetaldehyde, the selectivity may be at least 80% and defined as moles of acetaldehyde produced-'moles of ethanol consumed) *100.

The conversions and selectivities described above may be reported with reference to a particular set of conditions used in the method.

### EXAMPLES

### Example I

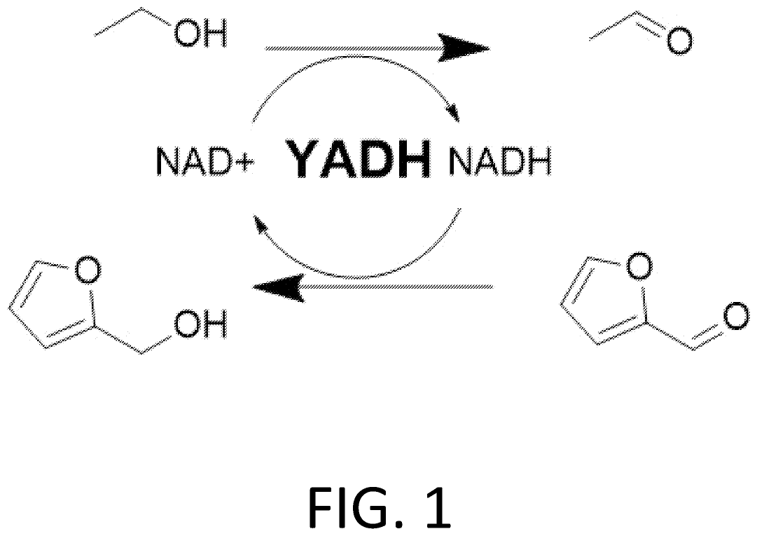

Experiments illustrating the co-conversion of ethanol to acetaldehyde and furfural to furfuryl alcohol were carried out as follows. A schematic representation of the biocatalytic redox cascade is shown in FIG. 1.

Various amounts of aqueous 200 mM sodium phosphate dibasic and monobasic buffer solutions were mixed to provide aqueous 100 mM sodium phosphate buffer solutions having various pHs, including pH 8.

A reactant mixture was prepared using the aqueous buffer solutions prepared above and amounts of furfural and ethanol to produce a final concentration of 20 mM furfural and 160 mM ethanol in the reactant solution.

Coenzyme β-NAD+ was added to the reactant mixture to produce a final concentration of 0.5 mM coenzyme.

The redox reaction was initiated by introducing a mixture comprising YADH1, YADH2, and YADH3 in various proportions to the reactant mixture (with coenzyme) such that the total enzyme concentration in the final reaction mixture (15 mL) was 0.2 mg/mL.

The final reaction mixture was incubated on an incubator shaker set to room temperature for 16 hours.

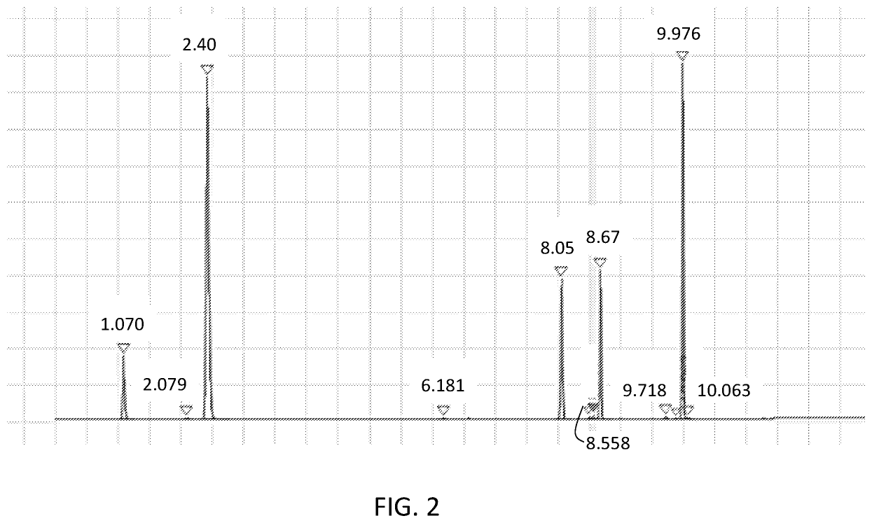

The products were analyzed using a gas chromatograph-flame ionization detector assembly fitted with a DB-WAXETR column. A typical chromatograph is shown in FIG. 2.

TABLE 1
Selectivity/conversion values.

| | | | | | | | | Furfuryl | |
|---|---|---|---|---|---|---|---|---|---|
| Enzyme | | Cofactor | Ethanol: | Ethanol | Furfural | Ethanol | Furfural | alcohol | |
| concentration | | concentration | Furfural | Concentration | concentration | conversion | conversion | selectivity | Acetaldehyde |
| (mg/ml) | pH | (mM) | ratio | (mM) | (mM) | (%) | (%) | (%) | selectivity (%) |
| 0.025 | 8 | 0.5 | 4:1 | 160 | 40 | 9.13 | 37.37 | 101.73 | 93.88 |
| 0.05 | 8 | 0.5 | 4:1 | 160 | 40 | 11.92 | 52.43 | 103.61 | 88.95 |
| 0.1 | 8 | 0.5 | 4:1 | 160 | 40 | 14.16 | 62.11 | 103.34 | 103.03 |
| 0.2 | 8 | 0.5 | 4:1 | 160 | 40 | 15.15 | 70.46 | 87.04 | 97.87 |
| 0.3 | 8 | 0.5 | 4:1 | 160 | 40 | 12.50 | 64.66 | 94.62 | 111.78 |
| 0.2 | 8 | 0.5 | 1:4 | 10 | 40 | 61.46 | 20.60 | 93.91 | 93.65 |
| 0.2 | 8 | 0.5 | 1:1 | 40 | 40 | 35.09 | 43.87 | 99.14 | 94.64 |
| 0.2 | 8 | 0.5 | 4:1 | 160 | 40 | 15.67 | 73.61 | 93.07 | 81.83 |
| 0.2 | 8 | 0.5 | 5:1 | 200 | 40 | 11.79 | 76.05 | 96.18 | 97.64 |
| 0.2 | 8 | 0.5 | 4:1 | 160 | 40 | 17.36 | 71.28 | 91.50 | 87.80 |
| 0.2 | 8 | 0.5 | 4:1 | 320 | 80 | 10.30 | 47.46 | 95.08 | 110.10 |
| 0.2 | 8 | 0.1 | 4:1 | 160 | 40 | 15.81 | 58.67 | 90.54 | 82.27 |
| 0.2 | 8 | 0.25 | 4:1 | 160 | 40 | 15.94 | 65.21 | 92.26 | 86.97 |
| 0.2 | 8 | 0.5 | 4:1 | 160 | 40 | 16.25 | 73.47 | 92.85 | 78.73 |
| 0.2 | 8 | 1 | 4:1 | 160 | 40 | 15.88 | 73.15 | 93.65 | 77.88 |
| 0.2 | 6 | 0.5 | 4:1 | 160 | 40 | 12.43 | 49.88 | 98.38 | 92.91 |
| 0.2 | 6.5 | 0.5 | 4:1 | 160 | 40 | 14.25 | 64.49 | 98.89 | 95.12 |
| 0.2 | 7 | 0.5 | 4:1 | 160 | 40 | 14.17 | 70.39 | 100.38 | 93.64 |
| 0.2 | 7.5 | 0.5 | 4:1 | 160 | 40 | 15.79 | 73.93 | 98.20 | 76.49 |
| 0.2 | 8 | 0.5 | 4:1 | 160 | 40 | 15.67 | 73.61 | 98.98 | 81.83 |
| 0.2 | 8.5 | 0.5 | 4:1 | 160 | 40 | 14.15 | 67.73 | 99.04 | 106.82 |
| 0.2 | 9 | 0.5 | 4.1 | 160 | 40 | 12.45 | 46.40 | 95.70 | 81.42 |

### Example 2

This example illustrates the simultaneous recovery of acetaldehyde from a product mixture comprising co-reactants and co-products. An aqueous product mixture having an initial volume of 202 mL: was formed by combining the following components: 0.15 M ethanol, 0.032 M acetaldehyde, 0.012 M furfural, 0.037 M furfuryl alcohol, and 0.1 M sodium phosphate buffer. A flowing stream of N₂ (>99.5% pure) was introduced into the product mixture under the following conditions; a gas flow rate of about 240 standard cubic centimeters per minute; a pressure of 1 atm; and a temperature of 22° C. The effluent gas was directed to a water column trap for collection. After 180 min, the product mixture had a final volume of 200 mL and contained 0.15 M ethanol, 0.014 M acetaldehyde, 0.012 M furfural, and 0.038 M furfuryl alcohol. Thus, the percent removal of the original moles of each component as a result of the simultaneous recovery was calculated to he 4.5% ethanol, 56% acetaldehyde, 4.2% furfural and −1.1% furfuryl alcohol (which is considered to be about zero within the error of the experiment).

### Example 3

This example demonstrates the immobilization of a yeast alcohol dehydrogenase (YADH) biocatalyst on a polymeric support and the characterization and use of the immobilized YADH biocatalyst in the coupled oxidation of ethanol to acetaldehyde and reduction of furfural to furfuryl alcohol.

The polymeric support used in this Example was Purolite® A110 (PA110), which is a primary amine functionalized macroporous polystyrene resin crosslinked with divinylbenzene. The particle size ranges between 0.3 mm and 1.2 mm. As received, the resin contained about 50-60% moisture. Thus, it was vacuum dried at 60° C. to remove the moisture and stored until use. Lyophilized yeast alcohol dehydrogenase (YADH) was obtained from Sigma Aldrich (the same source as used in Example 1) with a reported activity value of ˜300 U/mg. Coenzyme NAD+ was also obtained from Sigma Aldrich with a purity of ˜96%. All other chemicals were of analytical grade unless otherwise stated.

Resin moisture removal: As received, PA110 was weighed and vacuum dried at 60° C. for 24 hours. After 24 hours, the weight of the dried resin was recorded, and the moisture content was calculated.

Thermogravimetric analysis: Thermogravimetric measurements were done using an SDT Q600 TGA analyzer (TA instruments, PA, USA). A nitrogen purge flow was set at 100 ml/min with a constant heating ramp of 10° C. up to 600° C. and held at 600 ° C. for 15 mm.

Gas Chromatography: All reaction samples were analyzed by gas chromatography (GC) using an Agilent 7890B GC system fitted with a DB-WAXetr column and flame ionization detector assembly. Internal standardization was used for GC calibration and analysis, and diethylene glycol diethyl ether (DEGDEE) was used as the internal standard.

Sur face Area Analysis: Surface area analysis was done by nitrogen (N₂) physisorption using a Micromeritics ASAP 2020 Surface Area and Porosity Analyzer. The samples were degassed for 24 h at 30° C. under a vacuum to ensure the absence of undesirable gaseous molecules or water.

Fourier transform infrared spectroscopy (FT-IR) studies were performed using a Perkin Elmer Spectrum™FT-IR spectrometer with a standard optical system with KBr-window attachment. The unmodified and modified resin samples were ground with a mortar and pestle and mixed with ground KBr powder in a KBr: resin ratio of 20:1. Spectra were collected within a wavenumber range of 4000 cm⁻¹-500 cm⁻¹.

Enzyme (biocatalyst) concentration measurements: Enzyme concentration measurements were done using the Bradford assay. (Bradford. M. M. Analytical Biochemistry 1976, 72 (1), 248-254.) Briefly, 0.05 ml of enzyme solution in 0.1 M phosphate buffer was added to 0.95 ml of Coomassie Blue reagent, and the absorbance of the solution was measured at a wavelength of 595 nm using a Spectronic 200 spectrophotometer (Thermofisher Scientific). The calibration curve of absorbance vs. enzyme concentration was established within 0.1 mg/ml to 1 mg/ml YADH in 0.1 M sodium phosphate buffer.

Resin surface modification: 4 g of PA1.10 resin (dry basis) were activated by treating with 100 ml 0.025 M glutaraldehyde (GA) solution for 6 h to synthesize glutaraldehyde-activated PA110 (GA/PA110). The aldehyde groups of glutaraldehyde react with the primary amine groups on PA110 to form covalent linkages in the form of in-1′111e groups. (Barbosa, O. et al. Biomacromolecules 2013, 14 (8), 2433-2462) After treatment, analysis of the supernatant showed no glutaraldehyde. That is, all glutaraldehyde was adsorbed/reacted showing ˜6 mmol/g adsorbed on the resin. Remaining primary amine sites on the resin were capped with furfural (FAL) (the aldehyde group thereof which also reacts with a primary amine group to form an imine covalent linkage) to prevent these sites from interfering with subsequent enzymatic alcohol oxidation and FAL reduction reactions. Accordingly, the GA/PAT110 was treated with excess 0.5 M aqueous FAL solution for 16 h followed by water washing to synthesize FAL/GA/PA110. Analysis of the supernatant showed ˜5.4 mmol/g FAL was adsorbed/reacted. The total amount of primary amine sites (˜6 mmol/g) on the resin was calculated based on the amount of FAL and glutaraldehyde adsorbed by the resin using the following equation:

$$amine site density = \frac{( {C}_{GA , t = 0} - {C}_{Ga , t = 6 h} ) {V}_{GA} + ( {C}_{FAL , t - 0} - {C}_{FAL , t = 16 h} ) {V}_{n} - {\sum }_{i}^{n} {C}_{i} {V}_{i}}{{W}_{PA 110}}$$

wherein amine site density is the mmols of primary amines per unit mass of PA110 on dry weight basis (mmol/g), C_{GA,t=0}, C_{GA,t-6h}, C_{FAL,t=0}, and C_{FAL,t=16h} are the initial and final concentrations of the gluataraldehyde and FAL solutions respectively in mmol/L. V_{GA} and V_{FAL} are the volumes of the glutaraldehyde and FAL solutions used for adsorption. Ci and Vi are the concentration of FAL in the i^{th} wash solutions and volume of the i^{th} wash respectively.

Before immobilizing YADH, the FAL/GA/PA110 modified resin was washed three times with 60 ml 0.1 M phosphate buffer, followed by an ethanol (EtOH) wash of 20 ml. Any residual EtOH was removed by additional washing with water. All concentration measurements were done using gas chromatography with aliquots of samples taken from the supernatant solution before and after reactions with appropriate dilutions by the known volume of water addition.

YADH immobilization: The washed FAL/GA/PA110 modified resin (4 g dry basis) was added to a 100 ml solution of ˜0.5 mg/ml YADH in 0.1 M sodium phosphate buffer and set on an ice bath on a shaker for 16 h. Enzyme concentration in the supernatant before and after the immobilization were recorded to calculate enzyme loading (˜5.5 mg/g in this example) on the modified resin, using the following equation

$$enzyme loading ( \frac{mg}{g dry resin} ) = \frac{( {C}_{E , t = 0} - {C}_{E , t = 16 h} ) {V}_{E}}{{W}_{resin}}$$

wherein C_{E,t=0} and C_{E,t=16 h} are enzyme concentrations of the initial immobilization solution and final supernatant respectively in mg/ml. V_{g} is the volume of the immobilization solution in ml. Wᵣₑₛᵢₙ is the weight of the modified resin on a dry basis in g.

Residual activity test: To measure residual activity, NAD+ dependent EtOH oxidation was used as the activity assay. NAM appearance was observed and the concentration of NADH evolved was measured as a function of absorbance at 340 nm for 1 min in a total volume of 3 ml with known amount of the immobilized enzyme. All measurements were taken in a Cary 300 UV/vis spectrophotometer. YADH@FAL/GA/PA110 activity was calculated according to the following equation:

$$activity ( \frac{U}{mg} ) = \frac{\{ ( Δ {A}_{t = 1 min} - Δ {A}_{blank} ) \times 0.003 \}}{6.22 \times 1 \times enzyme loading \times {W}_{resin}} \times 1000$$

wherein ΔA_{t=1 min} and ΔA_{blank} are the absorbances of the assay mix after 1 min and blank solution (without enzyme),

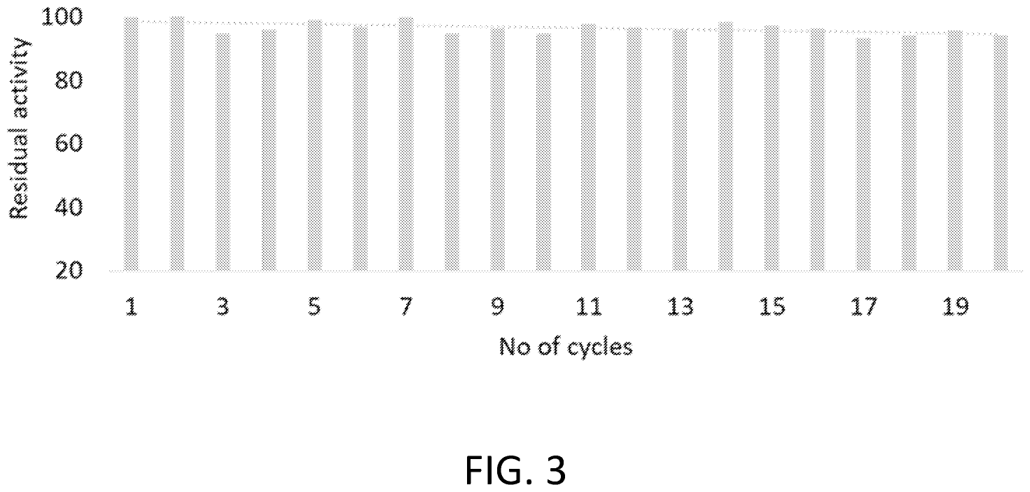

YADH@FAL/GA/PA110 activity in each cycle was calculated and platted as a percentage of the initial activity assay, as shown in FIG. 3. These results show that ˜94% enzyme activity was retained after 20 cycles and greater than 30 minutes.

Thermogravimetric Analysis: TGA plots were obtained for PA110 (unmodified resin) and YADH@FAL/GA/PA110 (modified resin with immobilized enzyme). After a week of storage, thermogravimetric analysis confirmed that the unmodified dried resin lost weight, but ultimately retaining about 20% moisture. The analysis showed that the weight loss rate was slower for YADH@FAL/GA/PA110 as compared to unmodified PA110.

Surface Area Analysis: Nitrogen physisorption studies were performed at 77 K and the BET surface area of PA110 and YADH@FAL/GA/PA110 were calculated. It was observed that the BET surface area (about 28 m²/g) was not affected by degassing temperature. It was also observed that the surface modification with glutaraldehyde, FAL. and enzyme immobilization did not affect the BET surface area.

Batch reactions: Batch redox reactions were run according to the reaction scheme shown in FIG. 1. Co-reactants (EtOH and FAL) were added to 101 ml of 0.1 M phosphate buffer aqueous solution to reach the desired initial concentrations. :A 1 ml aliquot of the solution was taken for initial concentration analysis. To the remaining solution, NAD+was added to reach a final concentration of 0.5 mM and 4 g YADH@FAL/GA/PA110 (dry basis) was added to initiate the redox reactions. The redox reactions were run in a closed vessel at room temperature on an incubator shaker for 4-16 h. A 1 ml aliquot was drawn at the end of reaction for final concentration analysis.

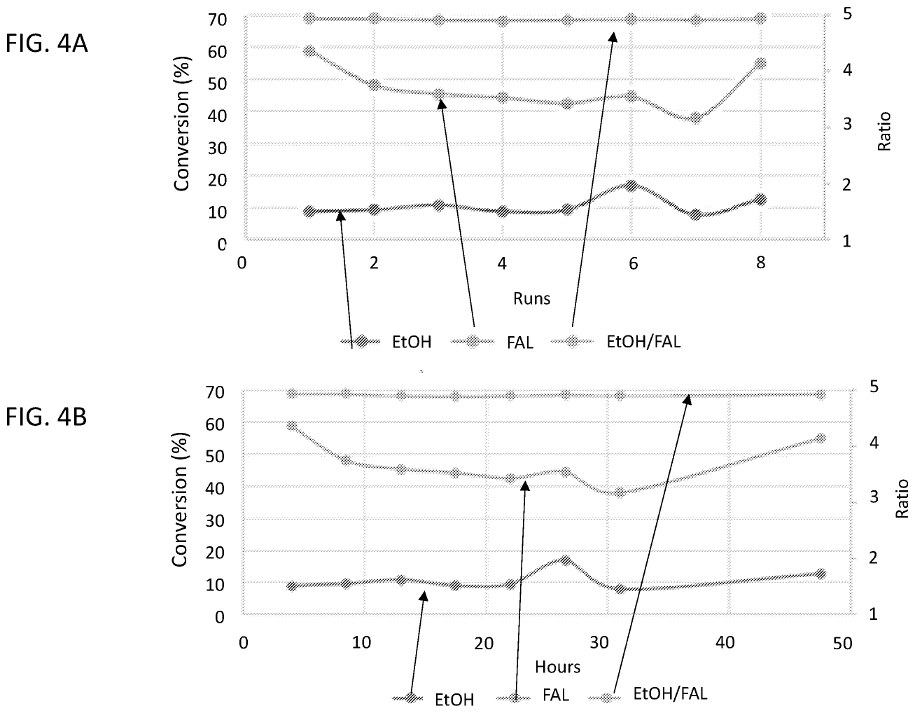

Eight batch reaction were performed, each batch running for 4 hours (except for the last batch running for 16 hours), with ˜30 minutes of washing between successive batches, corresponding to over 48 hours of continuous operation with a single batch of YADH@FAL/GA/PA110. The results are shown in FIGS. 4A and 4B. In the first hatch, a conversion of ˜59% FAL and ˜9% EtOH were obtained, In the next 6 consecutive runs, the conversion of FAL decreased to ˜40% while the EtOH conversion was maintained at ˜8%. The final batch reaction was run for 16 hours, with higher FAL and EtOH conversions of ˜55% and ˜13%, respectively, were observed.

Maximum substrate concentration: To test the resilience of the NADH@FAL/GA/PA110 towards deactivation by FAL, batch reactions were run with FAL concentrations between 37 mM and 72 mM. It was observed that FAL and EtOH conversions decreased consistently with the increase of initial substrate concentration.

The word “illustrative” is used herein to mean serving as an example, instance, or illustration. An aspect or design described herein as “illustrative” is not necessarily to be construed as preferred or advantageous over other aspects or designs. Further, for the purposes of this disclosure and unless otherwise specified, “a” or “an” means “one or more”.

If not already included, all numeric values of parameters in the present disclosure are proceeded by the term “about” which means approximately. This encompasses those variations inherent to the measurement of the relevant parameter as understood by those of ordinary skill in the art. This also encompasses the exact value of the disclosed numeric value and values that round to the disclosed numeric value.

The foregoing description of illustrative embodiments of the disclosure has been presented fir purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

What is claimed is:

1. A method for producing acetaldehyde from ethanol, the method comprising:
(a) exposing ethanol and furfural to a biocatalyst comprising yeast alcohol dehydrogenase 1, yeast alcohol dehydrogenase 2, and yeast alcohol dehydrogenase 3, and a biocatalyst cofactor under conditions that oxidize the ethanol to acetaldehyde and reduce the furfural to furfuryl alcohol to provide a product mixture comprising the acetaldehyde and the furfuryl alcohol; and
(b) recovering the acetaldehyde from the product mixture as it is being produced in step (a).

2. The method of claim 1, wherein step (b) is carried out by introducing an oxygen free gas to the product mixture under conditions to transfer at least a portion of the acetaldehyde from the product mixture to the gas to provide an effluent gas comprising the acetaldehyde, and collecting the effluent gas.

3. The method of claim 2, wherein the oxygen free gas comprises N₂.

4. The method of claim 2, wherein the oxygen free gas is introduced at a flow rate in a range of from 0.1 to 120 standard liters per minute per kilogram of the product mixture.

5. The method of claim 2, wherein the oxygen free gas has a pressure in a range of from 0.1 to 4 atm.

6. The method of claim 2, wherein the oxygen free gas comprises N₂, the oxygen free gas is introduced at a flow rate in a range of from 0.1 to 120 standard liters per minute per kilogram of the product mixture; and the oxygen free gas has a pressure in a range of from 0.1 to 4 atm.

7. The method of claim 1, wherein the biocatalyst is immobilized on a solid support.

8. The method of claim 7, wherein the solid support comprises a polymer and linking molecules covalently bound to surfaces of the solid support.

9. The method of claim 8, wherein the polymer is an amine-functionalized polystyrene and the linking molecules comprise glutaraldehyde.

10. The method of claim 1, wherein the ethanol, the furfural, or both are provided by a lignocellulosic biomass.

11. The method of claim 1, wherein step (b) is carried out by introducing an oxygen free gas to the product mixture under conditions to transfer at least a portion of the acetaldehyde from the product mixture to the gas to provide an effluent gas comprising the acetaldehyde, and collecting the effluent gas, and further wherein the biocatalyst is immobilized on a solid support.

12. The method of claim 11, wherein the solid support comprises a polymer and linking molecules covalently bound to surfaces of the solid support.

13. The method of claim 12, wherein the polymer is an amine-functionalized polystyrene and the linking molecules comprise glutaraldehyde.

14. The method of claim 13, wherein the oxygen free gas comprises N₂; the oxygen free gas is introduced at a flow rate in a range of from 0.1 to 120 standard liters per minute per kilogram of the product mixture; and the oxygen free gas has a pressure in a range of from 0.1 to 4 atm.

15. The method of claim 1, wherein the method provides a conversion of ethanol of at least 60%.

16. The method of claim 1, wherein the method provides a selectivity to acetaldehyde of at least 98%.

17. The method of claim 1, wherein the method provides a conversion of furfural of at least 75%.

18. The method of claim 1, wherein the method provides a selectivity to furfuryl alcohol of at least 98%.

19. The method of claim 1, wherein the method provides a conversion of ethanol of at least 9% and a conversion of furfural of at least 40% under continuous operation for at least 40 hours.
